(19) United States
(12) Patent Application Publication
(10) Pub. No.: US 2007/0032409 A1
(43) Pub. Date: Feb. 8, 2007

(54) Bradykinin receptor antagonists and uses thereof

(75) Inventors: Mias Pretorius, Nashville, TN (US); Nancy J. Brown, Nashville, TN (US); Laine J. Murphey, Nashville, TN (US)

Correspondence Address: FULBRIGHT & JAWORSKI, LLP, 1301 MCKINNEY, SUITE 5100, HOUSTON, TX, 77010-3095, US

(73) Assignee: VANDERBILT UNIVERSITY, Nashville, TN (US)

(21) Appl. No.: 11/341,395

(22) Filed: Jan. 26, 2006

Related U.S. Application Data
(60) Provisional application No. 60/647,166, filed on Jan. 26, 2005.

(51) Int. Cl. A61K 31/00; A61K 38/12; A61K 31/4745; A61K 31/66

(52) U.S. Cl. 514/9; 435/375; 514/114; 514/285; 514/291

(57) ABSTRACT

The present invention regards bradykinin receptor antagonists for treatment of a medical condition. In particular, the medical condition follows cardiopulmonary bypass. In specific embodiments, bradykinin receptor antagonists are used for the treatment of protamine-induced hypotension and/or fibrinolysis.

The present invention claims priority to U.S. Provisional Application Ser. No. 60/647,166, filed Jan. 26, 2005, which is incorporated by reference herein in its entirety.

### STATEMENT REGARDING FEDERALLY SPONSORED RESEARCH OR DEVELOPMENT

The present invention utilized funds from National Institutes of Health Grant No. NHLBI RO1 65193 and a Veterans Affairs Career Development Award to co-inventor M. Pretorius. The United States Government may have certain rights in the invention.

### FIELD OF THE INVENTION

The present invention generally concerns at least the fields of cell biology, molecular biology, anatomy, and medicine. Specifically, the field of the invention regards treatments related to cardiopulmonary bypass. In particular, the field of the invention concerns using bradykinin antagonists as therapy for hypotension and fibrinolysis.

### BACKGROUND OF THE INVENTION

Each year approximately 1 million patients undergo surgery requiring cardiopulmonary bypass (CPB) (Under and Ghosh, 2002). Large doses of heparin are used during CPB to prevent clot formation in the CPB circuit. Protamine, a highly basic polycationic polypeptide, is given at the end of CPB to reverse the anticoagulation effects of heparin. The administration of protamine is associated with several adverse effects including systemic hypotension, anaphylactoid reactions, and pulmonary vasoconstriction (Carr and Silverman, 1999). Protamine-induced hypotension is primarily the result of peripheral vasodilation (Shapira et al., 1982) that is mediated by the release of endothelial nitric oxide (NO) (Raikar et al., 1996; Viaro et al., 2002; Pearson et al., 1992). The mechanism underlying the stimulation of NO release from the endothelium is not known.

CPB leads to activation of the coagulation cascade (Hunt et al., 1998; Koster et al., 2002) with formation of factor XIIa that activates prekallikrein to kallikrein (Wendel et al., 1999). Kallikrein in turn cleaves bradykinin, a nonapeptide, from high molecular weight kininogen (HMWK) (Agostoni et al., 2001; Erdos, 2002). Bradykinin circulates with a half-life of seconds and causes vasodilation via its B2 receptor through increases in NO, prostacyclin (PGI2) and endothelium-derived hyperpolarizing factor (EDHF) (Vanhoutte, 1989). In addition to causing vasodilation, bradykinin stimulates the endothelial release of tissue-type plasminogen activator (t-PA) (Brown et al., 2000). The present inventors (Pretorius et al., 2004) and others (Cugno et al., 1999; Campbell et al., 2001) have reported that bradykinin concentrations increase during CPB.

Kimura et al. (2002) discloses that bradykinin increases expression of tissue factor and plasminogen activator inhibitor-1, and the increase in expression is suppressed by HOE 140.

Satake et al. (1996) regards pancreatitis-induced hypotension and the improvement of the hypotension in some animals receiving administration of HOE 140. In specific embodiments, HOE 140 inhibited the release of bradykinin and beta-endorphin.

In Griesbacher and Lembeck (1992), HOE-140 blocks the decreases in blood pressure following intravenous delivery of bradykinin, intravenous delivery of endogenously-released kinins, and the induction of acute pancreatitis in rats.

Christopher et al. (1994) concerns the alleviation of shock-induced hypotension with the bradykinin receptor antagonist CP-0127 following phenobarbital administration.

In Carini et al. (2002), the bradykinin receptor antagonists MEN11270 and HOE 140 increased blood pressure, which correlated with antagonism of bradykinin-induced hypotension.

Wang et al. (1997) refers to administration of HOE 140 restoring blood pressure to normal levels in hypotensive transgenic mice that harbor the B₂ receptor under the control of the Rous sarcoma virus 3′-LTR promoter.

Witherow et al. (2003) describes B9340 as a dose-dependent inhibitor of bradykinin-induced forearm vasodilitation and t-PA release.

U.S. Pat. No. 5,817,756 considers bradykinin receptor antagonists and, in specific embodiments, their use in bradykinin-induced hypotension.

WO 94/09001 regards bradykinin antagonists for mammals in need of treatment thereof.

### BRIEF SUMMARY OF THE INVENTION

In general, the present invention uses one or more bradykinin receptor antagonists for the treatment of a medical condition. The medical condition may be of any kind such that the bradykinin receptor antagonist is therapeutic, but in particular embodiments the medical condition is related to cardiac disease or treatments thereof. In specific embodiments, the medical condition is related to cardiopulmonary bypass. In particular, the medical condition is protamine-induced hypotension, fibrinolysis, or both, particularly in association with cardiopulmonary bypass. In additional embodiments, a medical condition that results as a direct or indirect use of protamine includes anaphylactic reaction and pulmonary hypertension, for example.

In specific embodiments of the invention, there is a method of reducing protamine-induced hypotension and/or fibrinolysis in an individual, comprising the step of delivering a bradykinin receptor antagonist to the individual. Although the bradykinin receptor antagonist may be delivered in any suitable manner, in specific embodiments, the bradykinin receptor antagonist is delivered to the individual intravenously, orally, intramuscularly, and so forth. In certain aspects of the invention, the bradykinin receptor is further defined as a B2 receptor.

The bradykinin receptor may be an indirect or direct target of the inhibitor, and it may be further defined as a B2 receptor. In particular aspects of the invention, the bradykinin receptor antagonist comprises HOE 140; CP-0127; MEN1 1270; NPC 18688; B9340; FR167344 (N-[N-[3-[(3-bromo-2-methylimidazo[1,2-a]pyridin-8-yl)oxymethyl]-2,4-dichlorophenyl]-N-methylaminocarbonylmethyl]-4-(dimethylaminocarbonyl)cinnamylamide hydrochloride); bradyzide; FR 173657; WIN 64338 ([[4-[[2-[[bis(cyclohexylamino)methylene]amino]-3-(2-naphthyl)-1-oxopropyl]amino]phenyl]methyl]tributylphosphonium chloride monohydrochloride)); B9858 (Glenn et al., 2002); NPC 18884 (Scios Nova, Inc.; Sunnyvale, Calif.); LF 16-0687 Ms (Kaplanski et al., 2002); Noscapine hydrochloride (3S)-6,7-Dimethoxy-3-[(5R)-5,6,7,8-tetrahydro-4-methoxy-6-methyl-1,3-dioxolo[4,5-g]isoquinolin-5-yl]-1 (3H)-isobenzofuranone hydrochloride; NPC 17731; D-Arg[Hyp3,Thi5,HypE(trans-propyl)7,Oic8]-BK or a mixture thereof, for example.

The individual being treated by bradykinin antagonists may be in need thereof, and in particular embodiments the individual will be having cardiopulmonary bypass, presently having cardiopulmonary bypass, previously having been subjected to cardiopulmonary bypass, or a combination thereof. In specific embodiments, the individual is subjected to an additional therapy, such as an additional cardiovascular disease therapy, hypotension therapy, and/or fibrinolytic therapy, for example.

In one embodiment of the invention, there is a method of reducing protamine-induced hypotension in an individual, comprising the step of delivering a bradykinin receptor antagonist to the individual. In particular aspects of the invention, the bradykinin receptor antagonist is delivered to the individual intravenously, orally, intramuscularly, or by inhalation, for example. In certain aspects of the invention, the bradykinin receptor is further defined as a B2 receptor.

The individual may be further defined as one to be subjected to extracorporeal circulation, as being subjected to extracorporeal circulation, or as previously subjected to extracorporeal circulation. In specific embodiments of the invention, the extracorporeal circulation comprises at least one of cardiopulmonary bypass, hemodialysis, or extracorporeal membrane oxygenation (ECMO), for example. In further embodiments, the individual is subjected to an additional therapy, such as administration of one or more drugs, blood transfusion, exercise, nutritional therapy, intravenous saline administration, or a combination thereof, for example. Exemplary drugs include anti-inflammatory drugs, anti-clotting drugs, or both, for example.

In another embodiment of the invention, there is a method of treating fibrinolysis, blood loss, or both in an individual, comprising the step of delivering a bradykinin receptor antagonist to the individual. In specific embodiments, the bradykinin receptor antagonist is delivered to the individual orally, intravenously, intramuscularly, or by inhalation, for example. The bradykinin receptor may be further defined as a B2 receptor.

In specific embodiments, the bradykinin receptor antagonist comprises HOE 140; CP-0127; MEN1 1270; NPC 18688; B9340; FR167344 (N-[N-[3-[(3-bromo-2-methylimidazo[1,2-a]pyridin-8-yl)oxymethyl]-2,4-dichlorophenyl]-N-methylaminocarbonylmethyl]-4-(dimethylaminocarbonyl)cinnamylamide hydrochloride); bradyzide; FR 173657; WIN 64338 ([[4-[[2-[[bis(cyclohexylamino)methylene]amino]-3-(2-naphthyl)-1-oxopropyl]amino]phenyl]methyl]tributylphosphonium chloride monohydrochloride)); B9858; NPC 18884; LF 16-0687 Ms (Kaplanski et al., 2002); Noscapine hydrochloride (3S)-6,7-Dimethoxy-3-[(5R)-5,6,7,8-tetrahydro-4-methoxy-6-methyl-1,3-dioxolo[4,5-g]isoquinolin-5-yl]-1(3H)-isobenzofuranone hydrochloride; NPC 17731; D-Arg[Hyp3,Thi5,HypE(trans-propyl)₇,Oic8]-BK; or a mixture thereof.

In further specific embodiments, the individual is further defined as to be subjected to extracorporeal circulation, as being subjected to extracorporeal circulation, as previously subjected to extracorporeal circulation, or a combination thereof. In particular aspects of the invention, extracorporeal circulation comprises cardiopulmonary bypass, hemodialysis, or extracorporeal membrane oxygenation (ECMO). The individual may be subjected to an additional therapy, such as administration of one or more drugs, blood transfusion, exercise, nutritional therapy, intravenous saline administration, or a combination thereof, in particular embodiments. Certain aspects of the invention regard the drugs as comprising anti-inflammatory drugs, anti-clotting drugs, or both.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 demonstrates arterial bradykinin concentrations before and after administration of protamine infusion. Error bars represent standard deviation.

FIG. 2 shows mean arterial pressure (MAP) response to protamine infusion in the saline and HOE 140 (bradykinin receptor antagonist) group. Nadir indicates the lowest MAP during protamine infusion. HOE 140 significantly blunted the decrease in MAP during protamine infusion (P=0.022, ANOVA). *P<0.05 versus pre-protamine, †P=0.001 versus HOE 140. Error bars represent standard deviation.

FIG. 3 shows tissue-type plasminogen activator (t-PA) activity in the saline and HOE 140 group. Baseline indicates measurement before initiation of cardiopulmonary bypass. HOE 140 significantly decreased t-PA activity compared to saline (−1.92±0.94 versus −0.68±1.41 IU/ml, P=0.036). *P<0.05 versus baseline, †P<0.05 versus intraoperative HOE 140, ‡P<0.05 versus Pre-protamine HOE 140. Error bars represent standard deviation.

### DETAILED DESCRIPTION OF THE INVENTION

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

The term “cardiopulmonary bypass” as used herein refers to circumvention (bypass) of the heart and lungs, such as in open heart surgery. A heart-lung machine (a pump-oxygenator) diverts blood returning to the heart before returning it to the arterial circulation. The machine performs the work both of the heart (pumping blood) and the lungs (supplying oxygen to red blood cells).

The term “fibrinolysis” as used herein refers to a process wherein one or more fibrin clots are broken down.

The term “hypotension” as used herein refers to abnormally low blood pressure. A skilled artisan recognizes that evaluation of hypotension depends on measurement of pressure and on decreased organ perfusion. In specific embodiments, a systolic blood pressure less than about 80 mmHg would be considered too low. In the present invention, protamine reduced mean arterial pressure is by about 20% in the presence of vehicle and about 6% in the presence of HOE 140.

The term “protamine” as used herein refers to protamine sulfate, which is indicated in the treatment of heparin use of overdosage.

The term “protamine-induced hypotension” as used herein refers to the indirect or direct result of hypotension occurring in an individual receiving protamine treatment or having received protamine treatment. In specific embodiments, the hypotension during and/or following protamine treatment is detected by any suitable method, although in specific embodiments it is detected by a sphygmomanometer, for example. In particular embodiments, however, blood pressure may be monitored by invasive means, such as with the case of patients undergoing cardiopulmonary bypass. A catheter is placed into an artery and connected to a fluid-filled transducer system that converts the pressure into an electronic signal that is displayed on a monitor. The system is calibrated, and pressure is displayed in mmHg.

II. The Present Invention

Arterial bradykinin increases significantly during cardiopulmonary bypass, and bradykinin concentrations remain elevated after protamine administration (Pretorius et al., 2004). In specific embodiments of the present invention, protamine reversal of heparin stimulates bradykinin release causing hypotension. In further specific embodiments, bradykinin mediates protamine-induced hypotension through the B2 receptor.

The present inventors conducted a double-blind randomized study in sixteen adult male patients undergoing elective cardiac surgery requiring CPB and taking an angiotensin-converting enzyme inhibitor preoperatively. Subjects were randomized to receive either saline (N=8) or the bradykinin B2 receptor antagonist HOE 140 (100 μg/kg, N=8) prior to the administration of protamine. Mean arterial pressure (MAP) and tissue-type plasminogen activator (t-PA) activity were measured intraoperatively and before and after protamine.

Protamine increased bradykinin concentrations from 6.3±4.3 to 12.2±9.8 finol/ml (P=0.006). Protamine significantly decreased MAP in the saline group, but bradykinin receptor antagonism blunted this effect. Hence, during protamine infusion, MAP was significantly lower in the saline group compared to the HOE 140 group. T-PA activity decreased significantly during HOE 140 administration, but not during saline. Similarly, t-PA activity decreased significantly during protamine administration in the HOE 140 group, but not in the saline group. Therefore, in specific embodiments of the present invention, increased levels of bradykinin contribute to protamine-induced hypotension through its B2 receptor. Administration of a bradykinin receptor antagonist comprises a novel therapy for the treatment and/or prevention of protamine-induced hypotension and fibrinolysis.

III. Bradykinin

The term “bradykinin” as used herein refers to a nonapeptide messenger that is enzymatically produced from kallidin. Its production may occur in the blood of an individual, and the half-life of bradykinin is brief, although it is an effective agent of arteriolar dilation and increased capillary permeability. Bradykinin is also released from mast cells during asthma attacks, from gut walls as a gastrointestinal vasodilator, from damaged tissues as a pain signal, and it may be a neurotransmitter. In exemplary embodiments, the sequence of human bradykinin may comprise the following sequence: RPPGFSPFR (SEQ ID NO:2).

The present invention concerns the inhibition of bradykinin for treatment of a medical condition. In particular aspects, at least one symptom of a medical condition is improved. In specific embodiments, this inhibition may employ inhibition of one or more of the bradykinin molecule, a receptor thereof, and a mediator that mediates binding of bradykinin to a receptor. The inhibition may be direct or indirect inhibition. The inhibition may be complete or partial.

In particular embodiments, bradykinin function or activity is inhibited, and this may be achieved by any appropriate means. For example, bradykinin may be inhibited by affecting the function, activity, half-life, or production of the molecule itself. The inhibition of bradykinin may comprise affecting its protein form, a DNA and/or RNA precursor form, or both. In particular embodiments, the inhibition of bradykinin affects its activity by affecting its binding to a receptor. In specific embodiments, the inhibition affects the ability of bradykinin to bind to a B2 receptor.

IV. Bradykinin Receptors

In specific embodiments, bradykinin receptors are cell surface receptors that bind bradykinin and related kinins with high affinity and trigger intracellular changes that influence the behavior of cells. The identified receptor types (B-1 and B-2, or BK-1 and BK-2, for example) recognize the endogenous kallidins, t-kinins, and certain bradykinin fragments as well as bradykinin itself.

An example of a bradykinin receptor includes bradykinin receptor 2, which is a G-protein coupled receptor, and in particular embodiments of the invention the bradykinin receptor antagonist acts through the B₂ receptor. Other names for the bradykinin receptor 2 include B₂ receptor BDKRB2, BKR2, B2 bradykinin receptor, BK2 receptor, or B2R, for example. In specific embodiments, it may also be referred to as BDKRB2 and comprises 44 kDa molecular weight. In further specific embodiments, the receptor comprises seven transmembrane domains. Binding of bradykinin to a receptor may be through direct or indirect binding.

B1 bradykinin receptor is another example of a bradykinin receptor, and it may be referred to as BDKRB1, Bradykinin receptor B1, BKR1, BK 1 receptor, B1R, or BRADYB1, for example. It also comprises seven transmembrane domains, has a molecular weight of 40 kDa, and is expressed in a greater number of tissues than the B₂ receptor.

V. Bradykinin Receptor Antagonists

Bradykinin receptor antagonists are used in the invention to ameliorate protamine-induced hypotension and/or fibrinolysis. Any bradykinin receptor antagonist that is suitable may be used in the invention. Furthermore, any antagonist that inhibits B2 receptor, B1 receptor, or both, may be employed in the invention, although in particular embodiments the antagonist inhibits B2 receptor. The antagonist may affect the function or activity of the receptor, the half-life of the receptor, the production of the receptor, or a combination thereof, for example.

In particular embodiments, the antagonists include one or more of the following: HOE 140; CP-0127; MEN1 1270; NPC 18688; B9340; FR167344 (N-[N-[3-[(3-bromo-2-methylimidazo[1,2-a]pyridin-8-yl)oxymethyl]-2,4-dichlorophenyl]-N-methylaminocarbonylmethyl]-4-(dimethylaminocarbonyl)cinnamylamide hydrochloride); bradyzide; FR 173657; WIN 64338 ([[4-[[2-[[bis(cyclohexylamino)methylene]amino]-3-(2-naphthyl)-1-oxopropyl]amino]phenyl]methyl]tributylphosphonium chloride monohydrochloride)); B9858 (Glenn et al., 2002); NPC 18884 (Scios Nova, Inc.; Sunnyvale, Calif.); LF 16-0687 Ms (Kaplanski et al., 2002); Noscapine hydrochloride (3S)-6,7-Dimethoxy-3-[(5R)-5,6,7,8-tetrahydro-4-methoxy-6-methyl-1,3-dioxolo[4,5-g]isoquinolin-5-yl]-1(3H)-isobenzofuranone hydrochloride; NPC 17731; D-Arg[Hyp3,Thi5,HypE(trans-propyl)₇,Oic8]-BK or mixtures thereof.

Furthermore, other bradykinin receptor antagonists may be employed such as, for example, those described in U.S. Pat. No. 5,817,756, incorporated by reference herein in its entirety. In general embodiments, these comprise compounds having, in appropriate spatial arrangement, two positively charged moieties flanking a hydrophobic organic moiety and a moiety that mimics a beta turn conformation. The compounds of the invention which interact with the B₂ receptors have the formula: X-Y-Z wherein, X is a moiety having a net positive charge; Y is a hydrophobic organic moiety having a nitrogen atom at the X-Y junction, a carbonyl group at the Y-Z junction, an atomic volume in the range of 135 Å³ to 300 Å³, and an end-to-end distance between the flanking nitrogen and carbonyl atoms of about 5.0Å±1.5 Å; Z is an arrangement of atoms which inherently adopt a beta turn conformation and has a positive charge near the distal end.

VI. Cardiopulmonary Bypass

Cardiopulmonary bypass refers to the use of the cardiopulmonary bypass machine to assume the native heart and lung function of the patient while the heart is arrested. Thus, cardiopulmonary bypass provides circulation and oxygenation of blood to maintain vital organ function. Cardiopulmonary bypass can sometimes also be referred to as extracorporeal circulation. Cardiopulmonary bypass, extracorporeal membrane oxygenation (ECMO), and hemodialysis (for renal failure) fall under the term extracorporeal circulation.

In many cases, CPB patients are given an anticoagulant (blood thinner), such as heparin, which helps to prevent massive extravascular coagulation (clotting of the blood while out of the body in the CBM) while the blood is circulating through the mechanical parts of the bypass system. Protamine is often administered to these patients to prohibit deleterious bleeding, and the present invention concerns amelioration of side effects therefrom.

VII. Protamine

Protamine may be referred to as protamine sulfate, although it may also be referred to as 013831 Formulation, particularly in relation to a manufacturer, Eli Lilly & Co. (Indianapolis, Ind.), and in specific embodiments comprises the following sequence: MARYRCCRSQ SRSRYYRQRQ RSRRRRRRSC QTRRRAMRCC RPRYRPRCRR H (SEQ ID NO:1). In specific embodiments, it is one of a class of simple proteins that are strongly basic, noncoagulable in heat, and yields diamino acids when hydrolyzed.

A skilled artisan recognizes that when administered alone, protamine has an anticoagulant effect, but when it is administered in the presence of heparin (which is strongly acidic), a stable salt is formed, resulting in the loss of anticoagulant activity of both drugs. Protamine has a rapid onset of action, with neutralization of heparin occurring within about 5 minutes after i.v. administration. In specific embodiments of the invention, the heparin-protamine complex stimulates bradykinin release, which then releases nitric oxide mediated through the B₂ receptor.

Protamine, such as in the form of protamine sulfate, may be administered in any suitable manner. In specific embodiments, protamine sulfate is given by very slow intravenous injection over a 10-minute period in doses not to exceed 50 mg. It is known that approximately each mg of protamine sulfate neutralizes approximately 90 USP units of heparin activity derived from lung tissue or about 115 USP units of heparin activity derived from intestinal mucosa. Because heparin disappears rapidly from the circulation, the dose of protamine sulfate required also decreases rapidly with the time elapsed following intravenous injection of heparin. For example, if the protamine sulfate is administered 30 minutes after the heparin, one half the usual dose may be sufficient.

A skilled artisan recognizes that it is important to keep an individual under close observation after cardiac surgery and receiving heparin and protamine. Additional doses of protamine may be administered if indicated by coagulation studies, such as a heparin titration test with protamine and the determination of plasma thrombin time, for example. In the event that deleterious effects manifest following protamine administration, the present invention provides a novel treatment therefor.

VIII. Fibrinolysis

Fibrinolysis is a process wherein a fibrin clot, such as one that is the product of coagulation, is broken down. Plasmin, a key player in the pathway, cuts the fibrin mesh at various places, leading to the production of circulating fragments that are cleared by other proteinases or by the kidney and liver. Plasmin is produced in an inactive form, plasminogen, in the liver. Although plasminogen cannot cleave fibrin, it still has an affinity for it, and is incorporated into the clot when it is formed.

Tissue plasminogen activator (tPA) is the agent that converts plasminogen to plasmin, thus allowing fibrinolysis to occur. tPA is released into the blood by the healthy endothelium in the areas immediately surrounding the clot. tPA is activated by thrombin and Factor XIIa. tPA itself is inhibited by other chemicals in the bloodstream. Plasminogen activator inhibitor 1 and 2 (PAI-1 and PAI-2) deactivate tPA.

The fibrinolysis may be a primary fibrinolysis, which occurs as an original condition, or a secondary fibrinolysis, which develops because of another disorder, medications, or other cause. In particular embodiments, the fibrinolysis is secondary fibrinolysis as a result of cardiopulmonary bypass, and in particular as a result of protamine administration therefor.

IX. Pharmaceutical Preparations

Pharmaceutical compositions of the present invention comprise an effective amount of one or more bradykinin receptor antagonists dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one bradykinin receptor antagonists or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The bradykinin receptor antagonist may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The bradykinin receptor antagonists may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include one or more bradykinin receptor antagonists, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the bradykinin receptor antagonist may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according tot he response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the bradykinin receptor antagonists are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, bradykinin receptor antagonists may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound bradykinin receptor antagonists may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

X. Combination Therapies

The present invention concerns treatments for protamine-induced hypotension and/or fibrinolysis, and in particular embodiments these conditions are associated with protamine treatment relating to cardiopulmonary bypass. In specific embodiments of the present invention, an individual receiving compositions of the present invention is further provided with an additional therapy. The additional therapy may comprise treatment for the protamine-induced hypotension, and/or fibrinolysis, cardiopulmonary bypass, cardiovascular disease, or combinations thereof, for example.

The treatment of the present invention may precede the additional therapy, it may be concomitant with the additional therapy, it may be subsequent to the additional therapy, or a combination thereof. An exemplary combination treatment regimen may be as follows. Various combinations may be employed, for example wherein the treatment of the present invention is “A” and the additional treatment, such as cardiovascular disease treatment, for example, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic compositions of the present invention to a patient will follow general protocols for the administration of drugs, taking into account the toxicity, if any, of the composition. It is expected that the treatment cycles would be repeated as necessary. It also is contemplated that various standard therapies, as well as surgical intervention, may be applied in combination with therapy.

The additional therapies that may be utilized in conjunction with the present invention may be of any kind suitable to the individual in need thereof. However, in specific embodiments the additional therapy may comprise administration of one or more drugs, blood transfusion, exercise, nutritional therapy, or a combination thereof, for example. In embodiments wherein an additional therapy is provided in the form of a drug, the drugs may comprise anti-inflammatory drugs, anti-clotting drugs, or a combination thereof.

Specific hypotension treatments include fludrocortisone; ibuprofen; desmopressin; octreotide; nutritional supplementation, such as with vitamin B12; and/or herbal supplementation, such as with hawthorn or panax ginseng, for example.

Exemplary treatments for fibrinolysis include aminocaproic acid, Aprotinin (Trasylol®) and Transexamic Acid (Cyclokapron®).

Examples of treatments for cardiovascular disease include Benazepril, such as Lotensin®; Benzthiazide, such as Aquatag® or Exna®; Beta-Blockers, such as Betapace®, Betimol®, Ophthalmic, Betoptic®, S Ophthalmic, Blocadren®, Lopressor®, Normodyne®, Ocupress®, Sectral®, Tenormin®, Timoptic®, OcuDose®, Timoptic®, Timoptic-XE®, Toprol XL®, Trandate®, or Zebeta®; Bumetanide, such as Bumex®; Captopril, such as Capoten®; Chlorothiazide, such as Diurigen® or Diuril®; Chlorthalidone, such as Lotensin®; Clonidine, such as Catapres® Oral, Catapres-TTS® Transdermal, or Duraclon® Injection, Enalapril, such as Vasotec® or Vasotec® I.V.; Fosinopril, such as Monopril®; Furosemide, such as Lasix®; Hydralazine, such as Apresoline®; Hydralazine & Hydrochlorothiazide, such as Apresazide®; Hydralazine, Hydrochlorothiazide, and Reserpine, such as Esidrix®, Ezide®, HydroDIURIL®, Hydro-Par®, Micro-zide™, and Oretic®; Hydrochlorothiazide, such as Esidrix®, Ezide®, HydroDIURIL®, Hydro-Par®, Microzide™, or Oretic®; Hydrochlorothiazide and Triamterene, such as Dyazide® or Maxzide®; Hydroflumethiazide, such as Diucardin® or Saluron®; Indapamide, such as Lozol®; Methyclothiazide, such as Aquatensen® or Endurong; Methyldopa, such as Aldomet®; Metolazone; Moexipril, such as Univasc®; Perindopril Erbumine, such as Aceon®; Polythiazide, such as Renese®; Potassium Chloride (Time Release), such as Cena-K®, Gen-K®, K+10®, Kaochlor®, Daochlor® SF, Kaon-Cl®, Kaon Cl-10®, Kay Ciel®, K⁺ Care®, K-Dur® 10, K-Dur® 20, K-Lease®, K-Lor™, Klor-Con®, Klor-Con® 8, Klor-Con® 10, Klor-Con/25®, Klorvess®, Klotrix®, K-Lyte/Cl®, K-Norm®, K-Tab®, Micro-Kg 10, Micro-K® Extencaps®, Micro-Kg LS®, Potasalan®, Rum-K®, Slow-K®, or Ten-K®; Quinapril, such as Accupril®; Quinethazone, such as Hydromox®; Ramipril, such as Altace™; Torsemide, such as Demadex®; Trandolapril, such as Mavik®; Triamterene, such as Dyrenium®; and Trichlormethiazide, such as Metahydrin® or Naqua®.

In order to increase the effectiveness of a bradykinin receptor antagonist, it may be desirable to combine these compositions and methods of the invention with an agent effective in the treatment of vascular or cardiovascular disease or disorder, in specific embodiments of the invention. In some embodiments, it is contemplated that a conventional therapy or agent, including but not limited to, a pharmacological therapeutic agent, a surgical therapeutic agent (e.g., a surgical procedure) or a combination thereof, may be combined with bradykinin receptor antagonist administration. Thus, in certain embodiment, a therapeutic method of the present invention may comprise administration of a bradykinin receptor antagonist of the present invention in combination with another therapeutic agent.

This process may involve contacting at least one cell(s) of the individual with an agent(s) and the bradykinin receptor antagonist at the same time or within a period of time wherein separate administration of the bradykinin receptor antagonist and an agent to a cell, tissue or organism of the individual produces a desired therapeutic benefit. The terms “contacted” and “exposed,” when applied to a cell, tissue or organism, are used herein to describe the process by which a therapeutic bradykinin receptor antagonist and/or therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both a bradykinin receptor antagonist and one or more agents, or by contacting the cell with two or more distinct compositions or formulations, wherein one composition includes a bradykinin receptor antagonist and the other includes one or more agents.

The bradykinin receptor antagonist may precede, be co-current with and/or follow the other agent(s) by intervals ranging from minutes to weeks. In embodiments where the bradykinin receptor antagonist, and other agent(s) are applied separately to a cell, tissue or organism, one would generally ensure that a significant period of time did not expire between the time of each delivery, such that the bradykinin receptor antagonist and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) as the bradykinin receptor antagonist. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1 week, about 2 weeks, about 3 weeks, about 4 weeks, about 5 weeks, about 6 weeks, about 7 weeks, about 8 weeks, about 1 month, about 2 months, about 3 months, about 4 months, about 5 months, about 6 months, about 7 months, about 8 months, about 9 months, about 10 months, about 11 months, or about 12 months, and any range derivable therein, prior to and/or after administering the bradykinin receptor antagonist.

Administration of the composition bradykinin receptor antagonist to a cell, tissue or organism may follow general protocols for the administration of vascular or cardiovascular therapeutics, taking into account the toxicity, if any. It is expected that the treatment cycles would be repeated as necessary. In particular embodiments, it is contemplated that various additional agents may be applied in any combination with the present invention.

A. Pharmacological Therapeutic Agents

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

1. Antihyperlipoproteinemics

In certain embodiments, administration of an agent that lowers the concentration of one of more blood lipids and/or lipoproteins, known herein as an “antihyperlipoproteinemic,” may be combined with administration of a bradykinin receptor antagonist for cardiovascular therapy, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thyroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, β-benzalbutyramide, camitine, chondroitin sulfate, clomestrone, detaxtran, dextran sulfate sodium, 5,8, 11, 14, 17-eicosapentaenoic acid, eritadenine, farazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

2. Antiarteriosclerotics

Non-limiting examples of an antiarteriosclerotic include pyridinol carbamate.

3. Antithrombotic/Fibrinolytic Agents

In certain embodiments, administration of an agent that aids in the removal or prevention of blood clots may be combined with administration of a bradykinin receptor antagonist for cardiovascular therapy, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemorrhage or an increased likelihood of hemorrhaging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine KI.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (nifedipine-type) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. Alpha Blockers

Non-limiting examples of an alpha blocker, also known as an □-adrenergic blocker or an β-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimethylline, trapidil, tricromyl, trimetazidine, troInitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, g-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

i. Arylethanolamine Derivatives

Non-limiting examples of arylethanolamine derivatives include amosulalol, bufuralol, dilevalol, labetalol, pronethalol, sotalol and sulfinalol.

ii. Benzothiadiazine Derivatives

Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

iii. N-carboxyalkyl(peptide/lactam) Derivatives

Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

iv. Dihydropyridine Derivatives

Non-limiting examples of dihydropyridine derivatives include amlodipine, felodipine, isradipine, nicardipine, nifedipine, nilvadipine, nisoldipine and nitrendipine.

v. Guanidine Derivatives

Non-limiting examples of guanidine derivatives include bethanidine, debrisoquin, guanabenz, guanacline, guanadrel, guanazodine, guanethidine, guanfacine, guanochlor, guanoxabenz and guanoxan.

vi. Hydrazines/Phthalazines

Non-limiting examples of hydrazines/phthalazines include budralazine, cadralazine, dihydralazine, endralazine, hydracarbazine, hydralazine, pheniprazine, pildralazine and todralazine.

vii. Imidazole Derivatives

Non-limiting examples of imidazole derivatives include clonidine, lofexidine, phentolamine, tiamenidine and tolonidine.

viii. Quanternary Ammonium Compounds

Non-limiting examples of quantemary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

ix. Reserpine Derivatives

Non-limiting examples of reserpine derivatives include bietaserpine, deserpidine, rescinnamine, reserpine and syrosingopine.

x. Suflonainde Derivatives

Non-limiting examples of sulfonamide derivatives include ambuside, clopamide, furosemide, indapamide, quinethazone, tripamide and xipamide.

7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

8. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene)or a miscellaneous diuretic such as aminozine, arbutin, chlorazanil, ethacrynic acid, etozolin, hydracarbazine, isosorbide, mannitol, metochalcone, muzolimine, perhexiline, ticmafen and urea.

c. Intropic Agents

Non-limiting examples of a positive intropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

9. Antianginal Agents

Antianginal agents may comprise organonitrates, calcium channel blockers, beta blockers and combinations thereof.

Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

10. Antibacterials

Antibacterials are generally used to reduce or prevent infection. Non-limiting examples of antibacterials include antibiotic antibacterials, synthetic antibacterials, leprostatic antibacterials rickettsia antibacterials, tuberculostatic antibacterial or a combination thereof.

a. Antibiotic Antibacterials

Non-limiting examples of antibiotic antibacterials include an aminoglycoside (e.g., amikacin, apramycin, arbekacin, a bambermycin, butirosin, dibekacin, dihydrostreptomycin, a fortimicin, gentamicin, isepamicin, kanamycin, micronomicin, neomycin undecylenate, netilmicin, paromomycin, ribostamycin, sisomicin, spectinomycin, streptomycin, streptonicozid, tobramycin), an amphenol (e.g., azidamfenicol, chloramphenicol, chlorampheniclol palmitate, chloramphenicol pantothenate, florfenicol, thiamphenicol), an ansamycin (e.g., rifamide, rifampin, rifamycin, rifaximin), a β-lactam (e.g., a carbapenem, a cephalosphorin, a cephamycin, a monobactam, an oxacephem, a penicillin), a lincosamide (e.g., clindamycin, lincomycin), a macrolide (e.g., azithromycin, carbomycin, clarithromycin, erythromycin acistrate, erythromycin estolate, erthromycin glucoheptonate, erythromycin lactobionate, erythromycin lactobionate, erythromycin propionate, erythromycin stearate, josamycin, leucomycin, midecamycin, miokamycin, oleandomycin primycin, primycin, rokitamycin, rosaramicin, roxithromycin, spiramycin, troleandomycin), polypeptides (e.g., amphomycin, bacitracin, capreomycin, colistin, enduracidin, enviomycin, fusagungine, a gramicidin, a gramicidin S, mikamycin, polymyxin, polymyxin B-Methanesulfonic acid, pristinamycin, ristoceitin, teicoplanin, thiostrepton, tuberactinomycin, tyrocidine, tyrothricin, vancomycin, viomycin, viomycin pantothenate, virginiamycin, zinc bacitracin), tetracycline (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, senociclin, tetracycline) or a micellaneous antibiotic antibacterial (e.g., cycloserin, mupirocin, tuberin).

Non-limiting examples of a carbapenem β-lactam include imipenem. Non-limiting examples of a cephalosporin β-lactam include cefaclor, cefadroxil, cefamandole, cefatrizine, cefazedone, cefazolin, cefixime, cefinenoxime, cefodizime, cefonicid, cefoperazone, ceforanide, cefotaxime, cefotiam, cefpimizole, cefpiramide, cefpodoxime proxetil, cefroxadine, cefsulodin, ceftazidime, cefteram, cftezole, ceftibuten, ceftizoxime, ceftriaxone, cefuroxime, cefuzonam, cephacetrile sodium, cephalexin, cephaloglycin, cephaloridine, cephalosporin C, cephalothin, cephapirin sodium, cephradine and pivcefalexin. Non-limiting examples of a cephamycin β-lactam include cefbuperazone, cefinetazole, cefminox, cefotetan and cefoxitin. Non-limiting examples of a monobactam β-lactam include aztreonam, carumonam and tigemonam. Non-limiting examples of a oxacephem β-lactam include flomoxef and moxolactam. Non-limiting examples of a penicillin β-lactam include amidinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carfecillin sodium, carindacillin, clometocillin, cloxacillin, cyclacillin, dicloxacillin diphenicillin sodium, epicillin, fenbenicillin, floxacillin, hetacillin, lenampicillin, metampicillin, methicillin sodium, mezlocillin, nafcillin sodium, mezlocillin, nafcillin sodium, oxacillin, penamecillin, penethamate hydridide, penicillin G benethiamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hhdrabamine, penimepicycline, phenethicillin potassium, piperacillin, pivampicillin, propicillin, quinacillin, sulbenicillin, talampicillin, temocillin and ticarcillin.

b. Synthetic Antibacterials

Non-limiting examples of synthetic antibacterials include 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantion), quinolones and quinone analogs (e.g., amifoxacin, cinoxacin, ciprofloxacin, difloxacin, enoxacin, fleroxacin, flumequine, lomefloxacin, miloxacin, nalidixic acid, norfloxacin, ofloxacin, oxolinic acid, pefloxacin, pipemidic acid, piromidic acid, rosoxacin, temafloxacin, tosulfoxacin), sulfonamides (e.g., acetyl sulfamehtoxypraxine, acetyl sulfisoxazole, azosulfamide, benzylsulfamide, choramine-B, chloramine-T, dichloramine T, formosulfathiazole, N2-formylsulfisomidine, N4-β-D-glucosylsulfanilamide, mafenide, 4′-(methylsulfanoyl)sulfanilamide, ρ-nitrosulfathiazole, phthalysulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, sulfanilamidomethanesulfonic acid triethanolamine salt, 4-sulfanilamidosalicylic acid, N4-sulfanilylsulfanilamide, sulfanilylurea, N-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole), sulfones (acedapsone, acediasulfone, acetosulfone sodium, dapsone, diathymosulfone, glucosulfone sodium, solasulfone, succisulfone, sulfanilic acid, p-sulfanilylbenzylamine, p,p′-sulfonyldianiline-N,N′diagalactoside, sulfoxone sodium, thiazolsulfone), and miscellaneous synthetic antibacterials (e.g., clofoctol, hexedine, methenamine, methenamine anhydromethylene-citrate, methenamine hippurate, methenamine mandelate, methenamine sulfosalicylate, nitroxoline, xibomol).

c. Liprostatic Antibacterials

Non-limiting examples of leprostatic antibacterials include acedapsone, acetosulfone sodium, clofazimine, dapsone, diathymosulfone, glucosulfone sodium, hydnocarpic acid, solasulfone, succisulfone and sulfoxone sodium.

d. Rickettsia Antibacterials

Non-limiting examples of rickettsia antibacterials, also known as antirickettsials, include p-aminobenzoic acid, chloramphenicol, chloramphenicol palmitate, chloramphenicol pantothenate and tetracycline.

e. Tuberculostatic Antibacterials

Non-limiting examples of tuberculostatic antibacterials include p-aminosalicylic acid, p-aminosalicylic acid hydrazine, benzoylpas, 5-bromosalicylhydroxamic acid, capreomycin, clofazimine, cyacetacide, cycloserine, dihydrostrptomycin, enviomycin, ethambutol, ethionamide, 4′-formylsuccinanilic acid thiosemicarbazone, furonazide, glyconiazide, isobutol, isoniazide, isoniazid methanesulfonate, morphazinamide, opiniazide, parsiniazide, phenyl aminosalicylate, protionamide, pyrazinamide, rifampin, salinazide, streptomycin, subathizone, sulfoniazide, thiacetazone, tiocarlide, tuberactinomycin, tubercidin, tuberin verazide, viomycin and vicmycin pantothenate.

B. Surgical Therapeutic Agents

In certain aspects, a therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Further treatment of the area of surgery may be accomplished by perfusion, direct injection, systemic injection or local application of the area with at least one additional therapeutic agent (e.g., a bradykinin receptor antagonist of the invention, a pharmacological therapeutic agent), as would be known to one of skill in the art or described herein.

XI. Screening For Modulators Of Bradykinin Receptor Function

The present invention further comprises methods for identifying modulators of the function of a bradykinin receptor, such as by a bradykinin receptor antagonist. These assays may comprise random screening of large libraries of candidate substances; alternatively, the assays may be used to focus on particular classes of compounds selected with an eye towards structural attributes that are believed to make them more likely to modulate the function of a bradykinin receptor.

By function, it is meant that one may assay for the ability to bind a bradykinin receptor, inhibit a bradykinin receptor, antagonize a bradykinin receptor, interfere with the binding of bradykinin to its receptor, and so forth.

To identify a bradykinin receptor modulator, one generally will determine the function of a bradykinin receptor in the presence and absence of the candidate substance, a modulator defined as any substance that alters function. For example, a method generally comprises:

(a) providing a candidate modulator;

(b) admixing the candidate modulator with an isolated compound or cell, or a suitable experimental animal;

(c) measuring one or more characteristics of the compound, cell or animal in step (c); and

(d) comparing the characteristic measured in step (c) with the characteristic of the compound, cell or animal in the absence of said candidate modulator,

wherein a difference between the measured characteristics indicates that said candidate modulator is, indeed, a modulator of the compound, cell or animal.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit or enhance bradykinin receptor activity. The candidate substance may be a protein or fragment thereof, a small molecule, or even a nucleic acid molecule. It may prove to be the case that the most useful pharmacological compounds will be compounds that are structurally related to an exemplary bradykinin receptor antagonist identified herein. Using lead compounds to help develop improved compounds is know as “rational drug design” and includes not only comparisons with known inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on a bradykinin receptor. Regardless of the type of inhibitor or activator identified by the present screening methods, the effect of the inhibition or activator by such a compound results in reducing protamine-induced hypotension and/or fibrolysis as compared to that observed in the absence of the added candidate substance.

B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

C. In cyto Assays

The present invention also contemplates the screening of compounds for their ability to modulate a bradykinin receptor in cells. Various cell lines can be utilized for such screening assays, including cells specifically engineered for this purpose.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

D. In vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that inhibits at least partially a bradykinin receptor. In these embodiments, the present invention is directed to a method for determining the ability of a candidate substance to bind a bradykinin receptor, generally including the steps of: administering a candidate substance to the animal; and determining the ability of the candidate substance to reduce one or more characteristics of the bradykinin receptor.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Exemplary Materials and Methods

Patients

Sixteen adult patients undergoing elective cardiac surgery requiring CPB and taking an ACE inhibitor preoperatively were studied. Patients were excluded if they had evidence of coagulopathy (INR greater than 1.7), underwent emergency surgery, underwent redo-CABG, had taken a glycoprotein IIb/IIIa antagonist within 2 days of surgery or had renal insufficiency (baseline creatinine>1.5 mg/dl). All patients provided written informed consent.

Protocol

The study protocol was approved by the Vanderbilt University and VA Tennessee Valley Healthcare System Institutional Review Boards and conducted according to the Declaration of Helsinki. Anesthesia management and CPB were conducted according to institutional protocol. Induction of anesthesia was achieved with either etomidate or thiopental and maintained with isoflurane, fentanyl, air and oxygen. Muscle relaxation was achieved and maintained with pancuronium or vecuronium. Invasive blood pressure monitoring was performed via either a radial or femoral arterial line (Arrow International, Reading, P A). A pulmonary artery catheter (Edwards Lifesciences, Irvine, Calif.) was placed in patients with a left ventricular ejection fraction less than 40%. CPB was achieved with a roller or centrifugal pump (Medtronic, Minneapolis, Minn.); heparin coated circuit (Carmeda®) and a Trillium® hollow fiber oxygenator (Medtronic, Minneapolis, Minn.). Heparin was used for anticoagulation during CPB at an initial dose of 400 U/kg supplemented with additional heparin to achieve and maintain an activated clotting time of >400s. The use of aprotinin and ε-aminocaproic acid was specifically excluded during this study. Pump prime consisted of 2000 ml of lactated ringers, 2000 U of heparin, 50 mmol/L of sodium bicarbonate and 500 mg of methylprednisolone. Eligible patients were randomized by the Vanderbilt Investigational Pharmacy to either the B2 receptor antagonist HOE 140 or saline. HOE 140 (100 μg/kg) or saline was administered as an intravenous bolus over one-half hour at the end of CPB but before protamine was given. The present inventors and others have shown that this dose of HOE 140 prevents vasodilation in response to exogenous bradykinin (Brown et al., 2000; Cockcroft et al., 1994). Protamine was given at an initial dose of 250 mg over 8 min; additional protamine, in 50 mg increments, was given if the ACT remained greater than 140s.

Vasopressors were used for separation from CPB according to the following criteria: left ventricular ejection fraction less than 40%, CPB time longer than 120 min or evidence of new onset left ventricular dysfunction by transesophageal echocardiography.

Patients were transfused according to the following guidelines: packed red blood cells (PRBC) were transfused for a hematocrit less than 20% during CPB and for a hematocrit less than 25% after CPB, CPB time >120 min, or evidence of end-organ dysfunction. Platelets were transfused in 5-U sets for ongoing microvascular bleeding despite a normalized ACT. Fresh frozen plasma (FFP) was given for continued bleeding only after platelets were given. Transfusion requirements were recorded from the beginning of surgery until hospital discharge.

Blood Sampling and Biochemical Assays

Arterial blood samples for measurement of bradykinin were collected prior to and after the administration of protamine. Arterial blood samples for measurement of t-PA activity, t-PA antigen and plasminogen activator inhibitor -1 (PAI-1) antigen were collected at four time points: 1) after induction of anesthesia and prior to CPB (baseline); 2) 60 min after initiation of CPB (intraoperative); 3) prior to administration of protamine (pre-protamine); 4) after administration of protamine (post-protamine). An additional blood sample was taken on postoperative day 1 for measurement of PAI-1 antigen. Arterial blood samples were taken from the CPB circuit during CPB and from an indwelling radial or femoral arterial line before and after CPB.

Blood for measurement of bradykinin was drawn into cold anhydrous ethanol and centrifuged after one hour; the supernatant was saved at −70° C. until the time of assay. Bradykinin was determined using a commercially available enzyme immunoassay (Peninsula Laboratories, Inc. Division of Bachem, San Carlos, Calif.). Blood for t-PA activity was collected in chilled vacutainer tubes containing acidified 0.105M sodium citrate (Biopool, Umea, Sweden) and centrifuged immediately at 0° C. for 20 minutes. Plasma was stored at −70° C. until the time of assay. PAI-1 and t-PA antigen levels were determined using a 2-site enzyme-linked immunosorbent assay (Immulyse, Biopool, Umea, Sweden) with results expressed as ng/ml. T-PA activity was measured using a chromogenic substrate and a standardized commercial kit (Chromolyse, Biopool Inc, Umea, Sweden), with results expressed as units/ml (IU/ml).

Statistical Analysis

Data are presented as means±standard deviations. Categorical baseline data were compared between groups using Chi-squared or Fischer's exact tests, as appropriate. Continuous data with non-parametric distribution were compared with the Wilcoxon Signed Rank test or the Mann-Whitney U test, as appropriate. Comparisons of the MAP, t-PA activity, t-PA antigen and PAI-1 antigen response between groups were made using a general linear model-repeated measures analysis of variance (ANOVA) in which the within-subject variable was time and the between-subject variable was administration of B2 receptor antagonist (saline versus HOE 140 group). A Bonferroni adjustment for multiple comparisons was done. A 2-tailed P-value less than 0.05 was considered statistically significant. All analyses were performed with the statistical package SPSS for Windows (Version 12.0, SPSS, Chicago, Ill.).

### Example 2

### Patient Characteristics

Table 1 provides the baseline characteristics of the patients randomized to saline or HOE 140. All subjects were male and had a history of hypertension.

TABLE 1
Patient Characteristics

| | Saline | HOE 140 | |
|---|---|---|---|
| | (N = 8) | (N = 8) | P-value |
| Age (years) | 57.3 ± 8.4 | 62.0 ± 10.3 | 0.330 |
| Race | 8:0 | 7:1 | 1.0 |
| (White:Nonwhite) | | | |
| Preoperative | 89.0 ± 17.1 | 81.4 ± 11.2 | 0.338 |
| MAP (mmHg) | | | |
| Heart Rate (bpm) | 56.1 ± 10.6 | 61.1 ± 15.5 | 0.488 |
| BMI (kg/M²) | 28.5 ± 4.7 | 29.2 ± 3.4 | 0.757 |
| Hematocrit (%) | 38.0 ± 5.9 | 37.6 ± 6.0 | 0.902 |
| Platelet Count | 260.0 ± 90.9 | 284.1 ± 149.7 | 0.734 |
| (K/μL) | | | |
| LVEF (%) | 46.0 ± 6.3 | 51.3 ± 10.6 | 0.248 |
| Active Smoker | 4(50) | 5(62.5) | 1.0 |
| N(%) | | | |
| Diabetes | 0(0) | 5(62.5) | 0.026 |
| Mellitus N(%) | | | |
| Hyperlipidemia | 7(87.5) | 6(75) | 1.0 |
| N(%) | | | |
| Aspirin N(%) | 6(75) | 8(100) | 0.467 |
| Beta-blockers | 7(87.5) | 8(100) | 1.0 |
| N(%) | | | |
| Calcium channel | 3(37.5) | 1(12.5) | 0.569 |
| blockers N(%) | | | |
| CABG:Valve | 6:2 | 6:2 | 1.0 |
| Procedure | | | |
| Number of grafts | 2.0 ± 1.3 | 1.8 ± 1.2 | 0.693 |
| Hemofiltration | 6(75) | 4(50) | 0.608 |
| N(%) | | | |
| Total Heparin | 42 875 ± 5890 | 43 500 ± 4928 | 0.821 |
| dose (Units) | | | |
| Total Protamine | 287 ± 88 | 300 ± 53 | 0.220 |
| dose (mg) | | | |

MAP indicates mean arterial pressure; BMI indicates body mass index; LVEF indicates left ventricular ejection fraction; CABG indicates coronary artery bypass graft.

There were no significant differences in age, race, MAP, heart rate, BMI, hematocrit, platelet count, left ventricular ejection fraction, smoking status, history of hyperlipidemia, preoperative aspirin use, beta-blockers use or calcium channel blocker use. There were significantly more patients with a history of diabetes in the HOE 140 group. There were no differences in the type of procedure, the number of grafts, the use of hemofiltration during CPB, total heparin dose or total protamine dose.

### Example 3

### Kinin Response to Protamine Administration

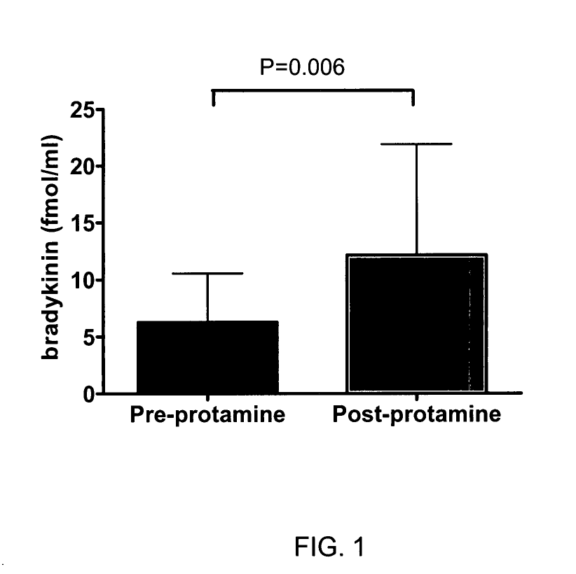

Protamine caused a significant increase in bradykinin concentrations (from 6.3±4.3 to 12.2±9.8 fmol/ml, P=0.006; FIG. 1). Bradykinin concentrations were similar in the saline and HOE 140 groups before protamine (6.1±3.6 versus 6.5±5.1 fmol/ml respectively, P=0.916) as well as after protamine administration (10.0±4.6 versus 14.3±13.1 fmol/ml, P=0.674).

### Example 4

### Hemodynamic Response to Protamine Administration

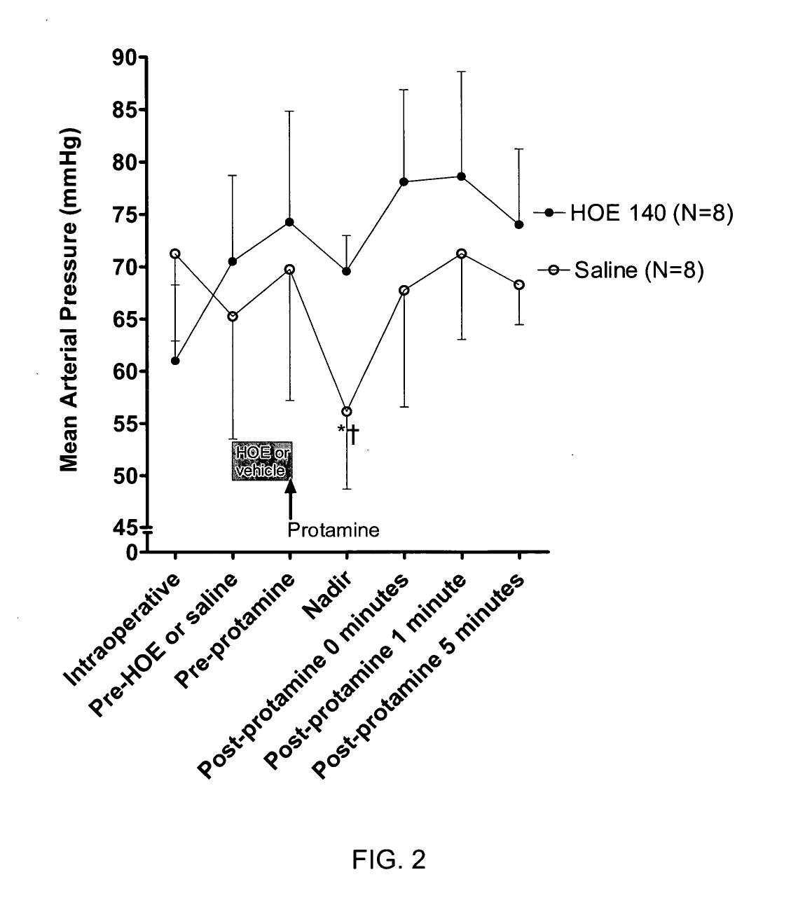

FIG. 2 indicates the hemodynamic response to HOE 140 and protamine administration in the two treatment groups. MAP was similar in the saline and HOE 140 groups at the start of the protamine infusion (69.8±12.5 versus 74.3±10.6 mmHg respectively, P=0.450). Infusion of protamine significantly decreased the MAP in the saline group (from 69.8±12.5 to 56.1±7.4 mmHg, P=0.031) but not in the HOE 140 group (from 74.3±10.6 to 69.6±3.4 mmHg, P=0.545). Hence, during protamine infusion MAP was significantly lower in the saline group compared to the HOE 140 group (56.1±7.4 versus 69.6±3.4 mmHg, P=0.001). MAP remained significantly lower after protamine infusion in the saline group compared to the HOE 140 group (F=4.794, P=0.046) for the remaining 5 minutes. There was no difference in heart rate between the saline and HOE 140 groups either prior to (82.4±13.8 versus 91.4±9.5, respectively, P=0.150) or after protamine (83.0±14.8 versus 81.0±14.0, respectively, P=0.705).

To exclude the possibility that differences in vasopressor use could account for differences in MAP in the 2 groups, the present inventors assessed the use of vasopressors during the time period of protamine administration (Table 2). There were no significant differences in the use of vasopressors or inotropes (all P=1.0 for use of norepinehrine, epinephrine, milrinone, dobutamine and dopamine) between the saline and HOE 140 groups during the administration of protamine.

TABLE 2
Vasopressor and inotropic support during
separation from cardiopulmonary bypass

| | Saline | HOE140 | |
|---|---|---|---|
| | (N = 8) | (N = 8) | P-value |
| Norepinephrine N(%) | 5(62.5) | 5(62.5) | 1.0 |
| Epinephrine N(%) | 2(25) | 2(25) | 1.0 |
| Milrinone N (%) | 1(12.5) | 0(0) | 1.0 |
| Dobutamine N(%) | 1(12.5) | 0(0) | 1.0 |
| Dopamine N(%) | 1(12.5) | 2(25) | 1.0 |
| 2 vasopressors N(%) | 2(25) | 2(25) | 1.0 |

### Example 5

### Fibrinolytic Response

As reported previously (Pretorius et al., 2003), PAI-1 antigen concentrations exhibited a biphasic response with an initial decrease during CPB (from 18.0±9.4 to 9.4±3.8 ng/ml, P=0.002) followed by an increase after surgery. PAI-1 antigen concentrations increased significantly during protamine administration in the HOE 140 group (from 20.2±17.1 to 27.4±15.5 ng/ml, P=0.006) but not in the saline group (from 17.9±13.3 to 19.1±9.2 ng/ml, P=0.746). PAI-1 antigen concentrations were similar in the two groups on postoperative day 1 (27.6±10.7 versus 32.4±10.1 ng/ml in the saline and HOE 140 group respectively, P=0.476).

Circulating t-PA antigen concentrations reflect both free active t-PA and t-PA complexed with PAI-1. Therefore, both t-PA activity and t-PA antigen were measured.

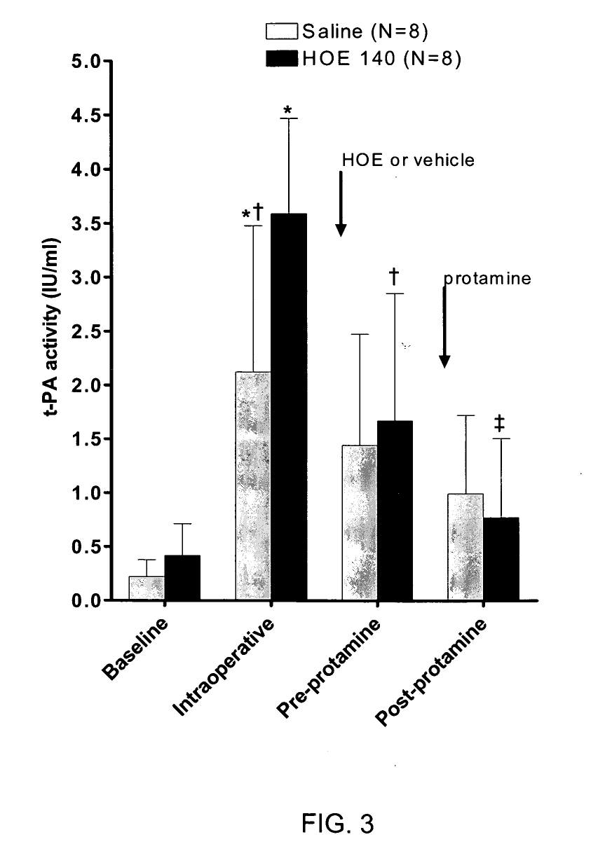

T-PA antigen did not change significantly in either group during study drug infusion (from 17.6±4.9 to 20.7±7.6 ng/ml, P=0.158 in saline group and from 23.4±12.6 to 20.1±6.1 ng/ml, P=0.440 in HOE 140 group) or during protamine administration (from 20.7±7.6 to 22.8±8.7 ng/ml, P=0.071 in saline group and from 20.1±6.1 to 22.7±9.1 ng/ml, P=0.229 in HOE 140 group). Baseline t-PA activity was similar in the saline and HOE 140 groups (0.22±0.16 versus 0.42±0.29 IU/ml respectively, P=0.093, FIG. 3). CPB caused a significant increase in t-PA activity (F=63.12, P<0.001). T-PA activity increased significantly during CPB in both the saline (from 0.22±0.16 to 2.12±1.36 IU/ml, P=0.004) and HOE 140 (from 0.42±0.29 to 3.59±0.88 IU/ml, P=<0.001) group; however, intraoperative t-PA activity (prior to receiving HOE 140 or saline) was significantly higher in the HOE 140 group compared to the saline group (F=7.64, P=0.015). As illustrated in FIG. 3, t-PA activity decreased significantly during HOE 140 administration (from 3.59±0.88 to 1.67±1.18 IU/ml, P=0.001) but not during saline (from 2.12±1.36 to 1.44±1.03 IU/ml, P=0.214). Similarly, t-PA activity decreased further during protamine infusion in the HOE 140 group (from 1.67±1.18 to 0.77±0.74 IU/ml, P=0.038) but not in the saline group (from 1.44±1.03 to 0.99±0.73 IU/ml, P=0.132).

TABLE 3
Safety
Intra- and Postoperative Patient Characteristics

| | Saline | HOE 140 | |
|---|---|---|---|
| | (N = 8) | (N = 8) | P-value |
| CPB time (min) | 122.0 ± 30.0 | 126.1 ± 46.2 | 0.835 |
| Cross-clamp time (min) | 94.6 ± 27.7 | 96.3 ± 43.4 | 0.930 |
| CT 4 hours (mL) | 338.6 ± 408.2 | 299.4 ± 233.0 | 0.817 |
| CT 24 hours (mL) | 1051.0 ± 1199.0 | 759.6 ± 276.4 | 0.514 |
| Time to extubation >10 | 1(12.5) | 2(25) | 1.0 |
| hours, N(%) | | | |
| PRBC transfusion | 2.9 ± 1.6 | 4.4 ± 4.0 | 0.488 |
| (Units) | | | |
| Platelet transfusion | 200 ± 0 | 275 ± 177 | 1.0 |
| (mL) | | | |
| Fresh frozen plasma | 0.1 ± 0.4 | 0.4 ± 1.1 | 0.927 |
| transfusion (Units) | | | |
| New onset atrial | 2(25) | 1(12.5) | 1.0 |
| fibrillation N(%) | | | |

CPB indicates cardiopulmonary bypass;
CT indicates chest tube output;
PRBC indicates pack red blood cells.

Table 3 provides the intra- and postoperative characteristics of the patients. There were no significant differences in chest tube output at 4 and 24 hours, number of patients in whom time to extubation was greater than 10 hours, total blood product transfusion or new onset atrial fibrillation. There was no mortality in either group at 30 days.

### Example 6

### Significance of the Present Invention

The administration of protamine causes hypotension in 2.9 to 11.2% (Seifert et al., 2003; Kimmel et al., 1998) of patients undergoing cardiac surgery requiring CPB. The mechanism of protamine-induced hypotension involves the release of NO (Raikar et al., 1996; Viaro et al., 2002; Pearson et al., 1992). Although protamine is a polypeptide rich in L-arginine, a precursor of NO, it is unlikely that protamine induces NO release by enhancing the provision of its substrate. Indeed protamine appears to inhibit L-arginine uptake into cells via specific cationic amino acid transport systems (Hammermann et al., 1999). Bradykinin stimulates endothelial NO synthase (Lauer et al., 2003). The present invention is the first demonstration that bradykinin contributes to protamine-induced hypotension through its B2 receptor.

Protamine administration significantly increased bradykinin concentrations. Although protamine administration has been shown to activate the classic complement pathway (Shastri et al., 1997) and histamine release (Patella et al., 1997), this is the first demonstration that protamine administration increases bradykinin concentrations in patients after CPB. Protamine inhibits carboxypeptidase N (kininase I) (Tan et al., 1989) and, thus, in specific embodiments increases bradykinin concentrations by decreasing the degradation of this nonapeptide. Protamine does not inhibit angiotensin-converting enzyme (ACE) or the neutral endopeptidase 24.11 or aminopeptidase M (Tan et al., 1989). While ACE constitutes the main enzyme responsible for bradykinin inactivation (88%), the contribution of carboxypeptidase N to the metabolism of bradykinin increases (from 12% to 46%) when ACE is inhibited (Cyr et al., 2001). In this regard, all the patients in the Examples were taking an ACE inhibitor prior to surgery and thus, in specific embodiments, the inhibitory effect of protamine on carboxypeptidase N may have had a greater impact on bradykinin concentrations than in patients not taking an ACE inhibitor. Protamine administration may also affect the generation of bradykinin by inducing local fluxes in charge (DeLucia et al., 1993) or by influencing the interaction between heparin and the bradykinin precursor HMWK (Bjojrk et al., 1989). Protamine (a positively-charged polypeptide) combines with heparin (a negatively-charged polypeptide) through an electrostatic interaction resulting in the formation of heparin-protamine complexes. In support of this latter mechanism, the vasodilator effects of protamine depend on the ratio of heparin and protamine (Oguchi et al., 2001). In specific embodiments of the present invention, the addition of protamine and heparin, but not protamine or heparin alone, to plasma generates bradykinin ex vivo.

Studies using the bradykinin B2 receptor antagonist HOE 140 have demonstrated that bradykinin causes vasodilation through its B2 receptor (Brown et al., 2000; Tramontana et al., 2001) and that endogenous bradykinin contributes to many of the effects of ACE inhibitors (Gainer et al., 1998; Pretorius et al., 2003). In the present invention, the finding that pretreatment with HOE 140 attenuated the hemodynamic response to protamine administration confirmed that increased endogenous bradykinin concentrations contributed to the hypotensive response following protamine. Moreover, the use of vasoconstrictors after CPB did not account for the differences observed in MAP response. This invention is the first use of a bradykinin receptor antagonist prior to protamine administration following CPB. Although the present inventors studied the contribution of bradykinin to protamine-induced hypotension, bradykinin is also generated during contact of blood with the CPB circuit during CPB (Pretorius et al., 2004; Campbell et al., 2001; Cugno et al., 2001). Studies utilizing the administration of bradykinin receptor antagonist early in CPB are needed to determine the contribution of endogenous bradykinin to the hypotensive, fibrinolytic and inflammatory effects of CPB. By analogy, a study of HOE 140 in an animal model of hemodialysis indicates that endogenous bradykinin contributes to hypotensive reactions to dialysis with negatively charged membranes during ACE inhibition (Krieter et al., 1998).

Bradykinin also stimulates t-PA release through its B2 receptor (Brown et al., 2000). Therefore, the present inventors determined the effect of HOE 140 on t-PA activity. Since t-PA is rapidly inactivated by complexing with PAI-1, and because concentrations of PAI-1 increase following CPB, decreases in t-PA activity at the end of CPB reflect the net balance between t-PA release and inactivation by complexing to PAI-1. The finding that t-PA activity decreased to a greater extent following HOE 140 administration than following saline may be attributed to the greater increase in PAI-1 antigen in the HOE 140 group compared to the saline group, in specific embodiments. Bradykinin decreases PAI-1 expression via its B2 receptor (Okada et al., 2004). However given the rapidity with which PAI-1 antigen concentrations increased following HOE 140 and protamine, it is unlikely that HOE 140 affected PAI-1 expression. Rather, bradykinin receptor blockade could have increased PAI-1 antigen by attenuating the inhibitory effect of NO on PAI-1 release from platelets (Korbut et al., 1995). Regardless of the mechanism, any effect of HOE 140 on fibrinolytic balance following protamine was short-lived as PAI-1 antigen concentrations were comparable on postoperative day (Unger et al., 2002). In specific aspects, bradykinin receptor blockade may attenuate the fibrinolytic response to CPB.

In summary, approximately 1 million people worldwide undergo CPB each year, and the reversal of heparin by protamine induces hypotension in 2.9 to 11.2% of these patients. The present invention indicates that increased bradykinin contributes to protamine-induced hypotension through the bradykinin B2 receptor. Administration of a bradykinin receptor antagonist is a novel therapy for the prevention of protamine-induced hypotension.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### PATENTS AND PATENT APPLICATIONS

U.S. Pat. No. 5,466,468

U.S. Pat. No. 5,580,579

U.S. Pat. No. 5,629,001

U.S. Pat. No. 5,641,515

U.S. Pat. No. 5,725,871

U.S. Pat. No. 5,780,045

U.S. Pat. No. 5,792,451

U.S. Pat. No. 5,817,756

WO 94/09001

### PUBLICATIONS

Agostoni A, Cugno M, Agostini A. Influence of contact system deficiencies during cardiopulmonary bypass. Thromb Haemost 2001;85(2):191-2.
Bjork I, Olson S T, Sheffer R G, Shore J D. Binding of heparin to human high molecular weight kininogen. Biochemistry 1989;28(3):1213-21.
Brown N J, Gainer J V, Murphey L J, Vaughan D E. Bradykinin stimulates tissue plasminogen activator release from human forearm vasculature through B₂ receptor-dependent, NO synthase-independent, and cyclooxygenase-independent pathway. Circulation 2000;102(18):2190-2196.
Campbell D J, Dixon B, Kladis A, Kemme M, Santamaria J D. Activation of the kallikrein-kinin system by cardiopulmonary bypass in humans. Am J Physiol Regul Integr Comp Physiol 2001;281(4):R1059-R1070.
Carini F, Guelfi M, Lecci A, Tramontana M, Meini S, Guiliani S, Montserrat X, Pascual J, Fabbri G, Ricci R, Quartara L, Maggi, C A. Cardiovascular effects of peptide kinin B₂ receptor antagonists in rats. Can. J. Physiol. Pharmacol. 2002; 80:310-322.
Carr J A, Silverman N. The heparin-protamine interaction. A review. J Cardiovasc Surg (Torino) 1999;40(5):659-66.
Christopher, T A, Ma, X-L, Gautheir, T W, Lefer, A M. Beneficial actions of CP-0127, a novel bradykinin receptor antagonist, in murine traumatic shock, Am. J. Physiol. 1994; 266:H867-H873.
Cockcroft J R, Chowienczyk P J, Brett S E, Bender N, Ritter J M. Inhibition of bradykinin-induced vasodilation in human forearm vasculature by icatibant, a potent B2-receptor antagonist. Br J. Clin Pharmacol 1994;38(4):317-21.
Cugno M, Nussberger J, Biglioli P, Alamanni F, Coppola R, Agostoni A. Increase of bradykinin in plasma of patients undergoing cardiopulmonary bypass—The importance of lung exclusion. Chest 2001;120(6): 1776-1782.
Cugno M, Nussberger J, Biglioli P, Giovagnoni M G, Gardinali M, Agostoni A. Cardiopulmonary bypass increases plasma bradykinin concentrations. Immunopharmacology 1999;43(2-3): 145-7.
Cyr M, Lepage Y, Blais C, Jr., et al. Bradykinin and des-Arg(9)-bradykinin metabolic pathways and kinetics of activation of human plasma. Am J Physiol Heart Circ Physiol 2001;281(1):H275-83.
DeLucia A, 3rd, Wakefield T W, Andrews P C, et al. Efficacy and toxicity of differently charged polycationic protamine-like peptides for heparin anticoagulation reversal. J Vasc Surg 1993;18(1):49-58; discussion 58-60.
Erdos E G. Kinins, the long march—a personal view. Cardiovasc Res 2002;54(3):485-91.
Gainer J V, Morrow J D, Loveland A, King D J, Brown N J. Effect of bradykinin-receptor blockade on the response to angiotensin-converting-enzyme inhibitor in normotensive and hypertensive subjects. New England Journal of Medicine 1998;339(18):1285-1292.
Glenn S. Mason, Michael J. Cumberbatch, Raymond G. Hill, and Nadia M. J. Rupniak. The bradykinin B1 receptor antagonist B9858 inhibits a nociceptive spinal reflex in rabbits, Can. J. Physiol. Pharmacol./Rev. Can. Physiol. Pharmacol. 2002; 80(4): 264-268.
Griesbacher, T, Lembeck, F. Effects of the bradykinin antagonist, HOE 140, in experimental acute pancreatitis, Br. J. Pharmacol. 1992; 107:356-360.

Hammermann R, Hirschmann J, Hey C, et al. Cationic proteins inhibit L-arginine uptake in rat alveolar macrophages and tracheal epithelial cells. Implications for nitric oxide synthesis. Am J Respir Cell Mol Biol 1999;21(2):155-62.
Hunt B J, Parratt R N, Segal H C, Sheikh S, Kallis P, Yacoub M. Activation of coagulation and fibrinolysis during cardiothoracic operations. Ann Thorac Surg 1998;65(3):712-8.
Hwang S J, Park H, Park K. Gastric retentive drug-delivery systems. Crit Rev Ther Drug Carrier Syst. 1998;15(3):243-84.
Kaplanski, J. et al. LF 16-0687 Ms, a Bradykinin B2 Receptor Antagonist, Reduces Brain Edema and Improves Long-Term Neurological Function Recovery after Closed Head Trauma in Rats, J. Neurotrama, August 2002, Vol. 19, No. 8, Pages 953-964.
Kimmel S E, Sekeres M A, Berlin J A, Ellison N, DiSesa V J, Strom B L. Risk factors for clinically important adverse events after protamine administration following cardiopulmonary bypass. J Am Coll Cardiol 1998;32(7):1916-22.
Kimura, S, Tsuiji, H, Nishimura, H., Kato, H., Ukimura, N., Yano, S., Kunieda, Y., Kawano, H., Nakagawa, K., Nakagawa, M. Bradykinin enhances in vitro procoagulant and antifibrinolytic properties of rat vascular endothelial cells. Thrombosis Research 2002; 106:41-50.
Korbut R, Marcinkiewicz E, Cieslik K, Gryglewski R J. The effect of nitric oxide donors on the release of plasminogen activator inhibitor (PAI) from rabbit platelets in vitro. J Physiol Pharmacol 1995;46(1):37-44.
Koster A, Fischer T, Praus M, et al. Hemostatic activation and inflammatory response during cardiopulmonary bypass: impact of heparin management. Anesthesiology 2002;97(4):837-41.
Krieter D H, Grude M, Lemke H D, et al. Anaphylactoid reactions during hemodialysis in sheep are ACE inhibitor dose-dependent and mediated by bradykinin. Kidney Int 1998;53(4):1026-35.
Lauer T, Kleinbongard P, Preik M, et al. Direct biochemical evidence for eNOS stimulation by bradykinin in the human forearm vasculature. Basic Res Cardiol 2003;98(2):84-9.
Mathiowitz E, Jacob J S, Jong Y S, Carino G P, Chickering D E, Chaturvedi P, Santos C A, Vijayaraghavan K, Montgomery S, Bassett M, Morrell C. Biologically erodable microspheres as potential oral drug delivery systems. Nature. 1997; 386(6623):410-4.
Oguchi T, Doursout M F, Kashimoto S, Liang Y Y, Hartley C J, Chelly J E. Role of heparin and nitric oxide in the cardiac and regional hemodynamic properties of protamine in conscious chronically instrumented dogs. Anesthesiology 2001;94(6):1016-25.
Okada H, Watanabe Y, Kikuta T, et al. Bradykinin decreases plasminogen activator inhibitor-1 expression and facilitates matrix degradation in the renal tubulointerstitium under angiotensin-converting enzyme blockade. J Am Soc Nephrol 2004;15(9):2404-13.
Patella V, Ciccarelli A, Lamparter-Schummert B, de Paulis A, Adt M, Marone G. Heterogeneous effects of protamine on human mast cells and basophils. Br J Anaesth 1997;78(6):724-30.
Pearson P J, Evora P R, Ayrancioglu K, Schaff H V. Protamine releases endothelium-derived relaxing factor from systemic arteries. A possible mechanism of hypotension during heparin neutralization. Circulation 1992;86(1):289-94.
Pretorius M, McFarlane J A, Vaughan D E, Brown N J, Murphey L J. Angiotensin-converting enzyme inhibition and smoking potentiate the kinin response to cardiopulmonary bypass. Clin Pharmacol Ther 2004;76(4):379-87.
Pretorius M, Murphey L J, McFarlane J A, Vaughan D E, Brown N J. Angiotensin-converting enzyme inhibition alters the fibrinolytic response to cardiopulmonary bypass. Circulation 2003:3079-3083.
Pretorius M, Rosenbaum D, Vaughan D E, Brown N J. Angiotensin-converting enzyme inhibition increases human vascular tissue-type plasminogen activator release through endogenous bradykinin. Circulation 2003;107(4):579-85.
Raikar G V, Hisamochi K, Raikar B L, Schaff H V. Nitric oxide inhibition attenuates systemic hypotension produced by protamine. J Thorac Cardiovasc Surg 1996;111(6):1240-6; discussion 1246-7.
Remington's Pharmaceutical Sciences, 18th Ed. Mack Printing Company, 1990.
Satake, K, Ha, S-S, Hiura, A. Effects of bradykinin receptor antagonist on the release of beta-endorphin and bradykinin and on hemodynamic changes in a canine model of experimental acute pancreatitis. Pancreas 1996; 12(1):92-97.
Seifert H A, Jobes D R, Ten Have T, et al. Adverse events after protamine administration following cardiopulmonary bypass in infants and children. Anesth Analg 2003;97(2):383-9, table of contents.
Shapira N, Schaff H V, Piehler J M, White R D, Still J C, Pluth J R. Cardiovascular effects of protamine sulfate in man. J Thorac Cardiovasc Surg 1982;84(4):505-14.
Shastri K A, Logue G L, Stem M P, Rehman S, Raza S. Complement activation by heparin-protamine complexes during cardiopulmonary bypass: effect of C4A null allele. J Thorac Cardiovasc Surg 1997;114(3):482-8.
Tan F, Jackman H, Skidgel R A, Zsigmond E K, Erdos E G. Protamine inhibits plasma carboxypeptidase N, the inactivator of anaphylatoxins and kinins. Anesthesiology 1989;70(2):267-75.
Tramontana M, Lecci A, Meini S, et al. Differences between peptide and nonpeptide B(2) bradykinin receptor antagonists in blocking bronchoconstriction and hypotension induced by bradykinin in anesthetized Guinea pigs. J Pharmacol Exp Ther 2001;296(3): 1051-7.
Unger F, Ghosh P. International cardiac surgery. Semin Thorac Cardiovasc Surg 2002;14(4):321-3.
Vanhoutte P M. Endothelium and control of vascular function: state of the art lecture. Hypertension 1989;13:658-667.
Viaro F, Dalio M B, Evora P R. Catastrophic cardiovascular adverse reactions to protamine are nitric oxide/cyclic guanosine monophosphate dependent and endothelium mediated: should methylene blue be the treatment of choice? Chest 2002;122(3): 1061-6.
Wang D, Chao L, Chao J. Hypotension in transgenic mice overexpressing human bradykinin B2 receptor. Hypertension 1997; 29(part 2):488-493.
Wendel H P, Jones D W, Gallimore M J. FXII levels, FXIIa-like activities and kallikrein activities in normal subjects and patients undergoing cardiac surgery. Immunopharmacology 1999;45(1-3): 141-4.

Witherow F N, Dawson P, Ludlam C A, Webb D J, Fox K A A, Newby D E. Bradykinin receptor antagonism and endothelial tissue plasminogen activator release in humans. Arterioscler Thromb Vasc Biol. 2003; 23:1667-1670.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of reducing protamine-induced hypotension in an individual, comprising the step of delivering a bradykinin receptor antagonist to the individual.

2. The method of claim 1, wherein the bradykinin receptor antagonist is delivered to the individual intravenously, orally, intramuscularly, or by inhalation.

3. The method of claim 1, wherein the bradykinin receptor antagonist is further defined as a B2 receptor antagonist.

4. The method of claim 1, wherein the bradykinin receptor antagonist comprises HOE 140; CP-0127; MEN11270; NPC 18688; B9340; FR167344 (N-[N-[3-[(3-bromo-2-methylimidazo[1,2-a]pyridin-8-yl)oxymethyl]-2,4-dichlorophenyl]-N-methylaminocarbonylmethyl]-4-(dimethylaminocarbonyl)cinnamylamide hydrochloride); bradyzide; FR 173657; WIN 64338 ([[4-[[2-[[bis(cyclohexylamino)methylene]amino]-3-(2-naphthyl)-1-oxopropyl]amino]phenyl]methyl]tributylphosphonium chloride monohydrochloride)); B9858; NPC 18884; LF 16-0687 Ms; Noscapine hydrochloride (3S)-6,7-Dimethoxy-3-[(5R)-5,6,7,8-tetrahydro-4-methoxy-6-methyl-1,3-dioxolo[4,5-g]isoquinolin-5-yl]-1(3H)-isobenzofuranone hydrochloride; NPC 17731; D-Arg[Hyp3,Thi5,HypE(trans-propyl)₇,Oic8]-BK; or a mixture thereof.

5. The method of claim 1, wherein said individual is further defined as to be subjected to extracorporeal circulation, as being subjected to extracorporeal circulation, or as previously subjected to extracorporeal circulation.

6. The method of claim 5, wherein the extracorporeal circulation comprises cardiopulmonary bypass, hemodialysis, or extracorporeal membrane oxygenation.

7. The method of claim 1, wherein said individual is subjected to an additional therapy.

8. The method of claim 7, wherein the additional therapy comprises administration of one or more drugs, blood transfusion, exercise, nutritional therapy, intravenous saline administration, or a combination thereof.

9. The method of claim 8, wherein the drugs comprise anti-inflammatory drugs, anti-clotting drugs, or both.

10. A method of treating fibrinolysis, blood loss, or both in an individual, comprising the step of delivering a bradykinin receptor antagonist to the individual.

11. The method of claim 10, wherein the bradykinin receptor antagonist is delivered to the individual orally, intravenously, intramuscularly, or by inhalation.

12. The method of claim 10, wherein the bradykinin receptor is further defined as a B2 receptor.

13. The method of claim 10, wherein the bradykinin receptor antagonist comprises HOE 140; CP-0127; MEN11270; NPC 18688; B9340; FR167344 (N-[N-[3-[(3-bromo-2-methylimidazo[1,2-a]pyridin-8-yl)oxymethyl]-2,4-dichlorophenyl]-N-methylaminocarbonylmethyl]-4-(dimethylaminocarbonyl)cinnamylamide hydrochloride); bradyzide; FR 173657; WIN 64338 ([[4-[[2-[[bis(cyclohexylamino)methylene]amino]-3-(2-naphthyl)-1-oxopropyl]amino]phenyl]methyl]tributylphosphonium chloride monohydrochloride)); B9858; NPC 18884; LF 16-0687 Ms; Noscapine hydrochloride (3S)-6,7-Dimethoxy-3-[(5R)-5,6,7,8-tetrahydro-4-methoxy-6-methyl-1,3-dioxolo[4,5-g]isoquinolin-5-yl]-1(3H)-isobenzofuranone hydrochloride; NPC 17731; D-Arg[Hyp3,Thi5,HypE(trans-propyl)₇,Oic8]-BK; or a mixture thereof.

14. The method of claim 10, wherein said individual is further defined as to be subjected to extracorporeal circulation, as being subjected to extracorporeal circulation, as previously subjected to extracorporeal circulation, or a combination thereof.

15. The method of claim 14, wherein said extracorporeal circulation comprises cardiopulmonary bypass, hemodialysis, or extracorporeal membrane oxygenation (ECMO).

16. The method of claim 10, wherein said individual is subjected to an additional therapy.

17. The method of claim 16, wherein the additional therapy comprises administration of one or more drugs, blood transfusion, exercise, nutritional therapy, intravenous saline administration, or a combination thereof.

18. The method of claim 17, wherein the drugs comprise anti-inflammatory drugs, anti-clotting drugs, or both.


SEQUENCE LISTING (2 sequences)

SEQ ID NO: 1 - protein, 51 aa, Artificial Sequence
  Description of Artificial Sequence  Synthetic Peptide
MARYRCCRSQSRSRYYRQRQRSRRRRRRSCQTRRRAMRCCRPRYRPRCRRH

SEQ ID NO: 2 - protein, 9 aa, Artificial Sequence
  Description of Artificial Sequence  Synthetic Peptide
RPPGFSPFR